(19) United States
(12) Patent Application Publication
(10) Pub. No.: US 2026/0256433 A1
(43) Pub. Date: Sep. 3, 2026

(54) METHOD FOR CONTROLLING AN X-RAY DEVICE, AN X-RAY DEVICE, AND A COMPUTER PROGRAM PRODUCT

(71) Applicant: Siemens Healthineers AG, Forchheim (DE)

(72) Inventor: Alois Regensburger, Poxdorf (DE)

(21) Appl. No.: 19/550,657

(22) Filed: Feb. 26, 2026

(30) Foreign Application Priority Data
Feb. 28, 2025 (DE) 10 2025 107 696.0

(51) Int. Cl. A61B 6/08; A61B 6/00; A61B 6/46

(52) U.S. Cl. CPC A61B 6/08; A61B 6/4441; A61B 6/461

(57) ABSTRACT

A method for controlling an X-ray device, wherein the X-ray device includes an X-ray source, an X-ray detector, and two light guide facilities arranged spaced apart from each other. The light guide facilities are configured to emit light fans that are rotatably mounted around the rotation axes. The method comprises receiving planning information for a planned path, positioning the arrangement, and moving the light fans based on the planning information and the current positioning so that the light fans illuminate the planned path.

### CROSS REFERENCE TO RELATED APPLICATIONS

This application claims the benefit of DE 10 2025 107 696.0 filed on Feb. 28, 2025, which is hereby incorporated by reference in its entirety.

### FIELD

Embodiments relate to a method for controlling an X-ray device, an X-ray device, and a computer program product.

### BACKGROUND

Precise placement of medical objects, such as needles or instruments, is often of critical importance in medical interventions. This applies for example to minimally invasive procedures like biopsies, pain therapies, or placing catheters. The medical object typically needs to be moved along a planned trajectory to a destination or oriented along a planned spatial direction. Imaging methods such as X-rays are often used to support such interventions, in order to visualize a position of the medical object in relation to an anatomy of a patient.

Conventional methods for guiding medical objects under X-ray control have various disadvantages, however. Systems for optical or electromagnetic tracking are often complex to use, may be susceptible to error, and require regular calibration. Additionally, their accuracy may be impaired by external influences, such as metallic objects or electromagnetic fields. Laser needle guidance systems on C-arm X-ray devices do enable visual support but are restricted in their flexibility. Precalculated views often have to be selected in the system menu, which slows down the workflow. Moreover, the provided needle guidance information is often only two-dimensional, which makes spatial orientation difficult.

Overall, these limitations result in complicated and less efficient guidance of medical objects under X-ray imaging control.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide improved three-dimensional guidance of medical objects under X-ray imaging control.

Embodiments relate in a first aspect to a method for controlling an X-ray device. The X-ray device includes an X-ray source and an X-ray detector opposite each other and two light guide facilities arranged spaced apart from each other, which are arranged in a first defined arrangement. The first defined arrangement is movably mounted. A first light guide facility of the two light guide facilities is configured to emit a first light fan and a second light guide facility of the two light guide facilities is configured to emit a second light fan. The first and the second light fan are at least rotatable around one rotation axis in each instance. The rotation axis of the first light fan intersects the rotation axis of the second light fan and a detector surface of the X-ray detector. In a first step, planning information is received for a planned path for arranging a medical object, which may be imaged for example by the medical X-ray device. In another step, the first light fan is emitted by the first light guide facility. In another step, the second light fan is emitted by the second light guide facility. In another step, the first defined arrangement is positioned in such a way that the rotation axis of the first light fan intersects the planned path. In another step, the first and/or the second light fan are moved in such a way, based on the planning information and a current positioning of the first defined arrangement, that the first and the second light fan illuminate the planned path. Moving the first and/or the second light fan includes at least one rotation of the first and/or the second light fan around the respective rotation axis.

The X-ray device may include the X-ray source and the X-ray detector. The X-ray source may be configured to emit X-rays, for example an X-ray beam. The X-ray detector may be configured to capture, for example detect, incidental X-rays, for example on an X-ray sensitive layer. The X-ray detector may be configured for example as a flat detector.

The X-ray source and the X-ray detector are arranged opposite each other. For example, the X-ray source and the X-ray detector are arranged opposite each other in such a way that the X-rays emitted by the X-ray source illuminate the X-ray detector, for example the X-ray sensitive layer of the X-ray detector. The X-ray source, the X-ray detector, and the two light guide facilities are also arranged in the first defined arrangement. The first defined arrangement may thereby indicate a relative positioning, for example a spatial relative position, and/or relative orientation, and/or relative pose, between the X-ray source, the X-ray detector, and the two light guide facilities. The X-ray source, the X-ray detector, and the two light guide facilities may also be arranged on a common support structure, for example, a C-arm, and/or C-arc, and/or O-arm, in the first defined arrangement. The common support structure may be movably mounted around one or more axes.

The first defined arrangement is movably mounted, for example translatably and/or rotatably mounted.

The X-ray device includes two light guide facilities arranged spaced apart from each other. The light guide facilities may advantageously each include a light source, for example, a laser light source and/or a laser module, which is configured to emit the respective light fan. The light guide facilities may include an optical collimator, lenses, mirrors, prisms, fiber optics, or combinations thereof for this purpose.

The first light guide facility of the two light guide facilities is configured to emit the first light fan. The second light guide facility of the two light guide facilities is configured to emit the second light fan. A light fan may include a flat diffusion of light, which starts from the light source and spreads in a certain direction. The light fans may, for example, be produced in each instance by a lens, a collimator, a slit, or another optical apparatus. The two light guide facilities, for example laser modules, may each, for example, be configured as line lasers, wherein the light emitted by the line lasers always spans a light fan in the space, which always lies within a plane.

The first and the second light fan are at least rotatable around one rotation axis in each instance. For example, the first and the second light fan may be at least rotatably mounted around one rotation axis in each instance. The rotatability of the light fans may be realized through mountings, for example, mechanical apparatuses, for example joints, bearings, and/or motorized systems, and/or electromagnetic apparatuses.

The rotation axis of the first light fan intersects the rotation axis of the second light fan and a detector surface of the X-ray detector, for example simultaneously. The detector surface of the X-ray detector may be configured as an area, for example a surface and/or plane, of the X-ray detector, which runs along or parallel to a flat side of the X-ray sensitive layer of the X-ray detector. For example, the detector surface may be adjacent to the X-ray sensitive layer of the X-ray detector, for example it may contact the X-ray sensitive layer, or run at least partially, for example completely, within the X-ray sensitive layer. When the X-ray detector has a curved form, for example the X-ray sensitive layer of the X-ray detector, the detector surface may run correspondingly curved, for example following a curve of the X-ray detector, for example a curve of the X-ray sensitive layer. The detector surface of the X-ray detector being intersected by the rotation axis of the first light fan may include a virtual extension of the detector surface, for example the plane, being intersected by the rotation axis of the first light fan. The virtual extension of the detector surface may thereby be arranged outside the X-ray detector, for example directly adjacent to the X-ray detector.

Receiving the planning information may include capturing and/or reading from a computer-readable data memory and/or receiving from a data memory unit, for example, a database. Furthermore, the planning information may be provided by a provision unit of one or more medical imaging facilities, for example using the same or different imaging modalities. Receiving the planning information may be effected by a control unit of the X-ray device. The control unit may, for example, include a computing unit, a memory unit, and/or an interface, which are configured to receive and process the planning information.

Advantageously, the planning information may have spatially, for example spatially and temporally, resolved information on a planned path, for example a planned needle path, for arranging the medical object. The planned path may run in a straight line at least in sections, for example completely. For example, the planning information may be provided using a pre-procedural data set, for example a pre-procedural 3D data set, of an examination object. The planning of the planned path may thereby be effected in an image data set taken by the X-ray device, for example. Alternatively or additionally, the planning of the planned path may be effected in a registered image data set. A planned path, for example a needle path, may have a start point and an end point. The planned path includes both the planned path itself and a linear extension of the planned path in both spatial directions.

The medical object may, for example, be configured as an, for example elongated, surgical and/or diagnostic instrument. For example, the medical object may be configured as flexible and/or rigid at least in sections. The medical object may, for example, be configured as a needle, for example a puncture needle, and/or catheter, and/or endoscope, and/or guide wire.

The examination object may, for example, be a human and/or animal patient and/or an examination phantom.

The emission of the first light fan by the first light guide facility and the emission of the second light fan by the second light guide facility may be understood as separate steps, in which the respective light guide facility is activated to produce the corresponding light fan and project it into the space. The steps for emitting the two light fans do not necessarily have to be effected in this order. The first and the second light fan are emitted at least occasionally at the same time or temporally interleaved. When the emission of the first and/or the second light fan is temporally interleaved, an interleaving frequency of the emission may advantageously lie outside a perception threshold of an observer, for example above 25 Hz. The emission of the first and second light fan may be controlled by the respective light guide facilities.

The emission of the two light fans may also be spatially interleaved. For example, the first and the second light fan may each be emitted as an, for example strip-shaped and/or dotted, light pattern, wherein the projected light patterns of the first and the second light fan interact along the planned path, for example having appropriate spatial interleaving.

The first and/or the second light guide facility may also be configured to emit the respective light fan having a light pattern, for example, a dot pattern, and/or having optically distinct angular ranges, for example, having a different light color, and/or brightness, and/or intensity. Alternatively or additionally, the first and/or the second light guide facility may also be configured to emit the respective light fan having a predefined angular range, for example within a predefined fan angle. The predefined light pattern, the optically distinct angular ranges, and/or the predefined angular range of the first and/or second light fan may, for example, indicate a target positioning for a planned arrangement of the medical object, for example a marker structure of the medical object, along the planned path, for example, a planned penetration depth of the medical object. Advantageously, the predefined light patterns, the optically distinct angular ranges, and/or the predefined angular range of the first and/or second light fan may be adjusted depending on the planning information and/or the current positioning of the first defined arrangement. For example, the first and/or the second light guide facility may be configured to adjust the predefined light pattern, the optically distinct angular ranges, and/or the predefined angular range of the first and/or second light fan depending on the planning information and/or the current positioning of the first defined arrangement.

Positioning the first defined arrangement may include moving, for example translating and/or rotating, the first defined arrangement. The first defined arrangement may be positioned in such a way that the rotation axis of the first light fan intersects the planned path. A movement unit of the X-ray device may be used to position the first defined arrangement. The movement unit may include motorized axes or other mechanisms, which provide precise movement and orientation of the X-ray source, the X-ray detector, and the two light guide facilities.

The first defined arrangement may, for example, be positioned by a calibrated movement unit, for example, a calibrated motor. The calibrated movement unit may thereby be configured to position the first defined arrangement and to provide the current positioning of the first defined arrangement. The calibrated movement unit may, for example, include a sensor for this purpose, for example an encoder, which is configured to capture a current configuration, for example a current positioning, of the calibrated movement unit. The current positioning of the first defined arrangement may advantageously be provided depending on the current positioning of the calibrated movement unit.

Alternatively or additionally, capturing the current positioning of the first defined arrangement may include measuring, and/or recording, and/or detecting a current spatial position, orientation, and/or pose of the first defined arrangement, for example of the X-ray source, the X-ray device, and the two light guide facilities. The current positioning of the first defined arrangement may be captured by a sensor, encoder, and/or a position detection system. The sensor, encoder, and/or the position detection system may be arranged on the X-ray device, for example on moving parts of the X-ray device, and/or spaced apart from the X-ray device.

The current positioning of the first defined arrangement may be captured by one or more of the following elements: optical sensors, for example, cameras and/or laser scanners, which may be configured to capture markings and/or reference points on the first defined arrangement, inertial measuring units (IMUs), which may be configured to measure accelerations and/or rotation rates and to derive the current positioning of the first defined arrangement therefrom, (electro)magnetic sensors, which may be configured to detect changes in a magnetic field and to capture the current positioning by these mechanisms, rotation encoders, which may be configured to precisely measure rotational movements of axes and/or joints of the first defined arrangement, encoders, which may be configured to capture translation movements and the current positioning along defined axes, ultrasound-based position detection systems, which may be configured to use runtime measurements of sound waves to determine position, optical tracking systems including one or more infrared cameras and reflective markers, wherein the at least one infrared camera or the markers may be arranged on the first defined arrangement, wherein the at least one infrared camera may be configured to capture the position of the markers and hereby the current positioning of the first defined arrangement, radar-based sensors, which may be configured to use electromagnetic waves to capture the current positioning, for example, by detecting changes in a reflection and runtime of the emitted radar waves, which are reflected by the first defined arrangement, electromagnetic tracking systems, which may be configured to produce a weak magnetic field and to determine the position of sensors in this field, wherein the sensors may be arranged on the first defined arrangement, hybrid systems, which may be configured to combine several of the named technologies, in order to achieve higher accuracy and/or robustness.

These sensors, encoders, and systems may be used individually or in combination, in order to ensure the highest possible accuracy and reliability when capturing the current positioning of the first defined arrangement.

Moving the first and/or the second light fan based on the planning information and the current positioning of the first defined arrangement may include adjusting the orientation and/or position of the light fans. Moving the first and/or the second light fan may include at least one rotation of the first and/or the second light fan around the respective rotation axis. The movement of the light fans, for example the rotation of the light fans, may be enabled by a respective movement element. The respective movement element may, for example, include a motorized system, for example an electromagnetic actuator, a piezoelectric drive, and/or a hydraulic system, and/or an adjustable optical element, for example a mirror and/or a prism. This advantageously enables precise control of the orientation of the light fans. The movement of the first and/or second light fan may be controlled by the control unit. The control unit may calculate the necessary rotation angle based on the planning information and the current positioning and provide corresponding control signals to the respective movement element.

The rotation axis of the first light fan may run within the layer, for example the plane, that is illuminated by the first light fan. Alternatively or additionally, the rotation axis of the first light fan may run through the first light guide facility, for example an exit point of the first light fan. Alternatively, the rotation axis of the first light fan may intersect the first light fan, for example may be arranged not parallel to the first light fan.

The rotation axis of the second light fan may run within the layer, for example the plane, that is illuminated by the second light fan. Alternatively or additionally, the rotation axis of the second light fan may run through the second light guide facility, for example an exit point of the second light fan. Alternatively, the rotation axis of the second light fan may intersect the second light fan, for example may be arranged not parallel to the second light fan.

The rotation axis of the first light fan thereby intersects the detector surface of the X-ray detector and the rotation axis of the second light fan.

The first and/or the second light fan are moved in such a way based on the planning information and the current positioning of the first defined arrangement that the first and the second light fan illuminate, for example simultaneously, the planned path. The two light fans may thereby have a common intersection line along the planned path, for example because of the spaced arrangement of the two light guide facilities. The intersection line, for example a crossing line, of the two light fans may thereby be oriented along the planned path. By arranging the light guide facilities spaced apart, the light fans may meet the planned path from different angles. When the light fans are arranged so that they intersect along the planned path, a clearly visible intersection line may arise. While an individual light fan may only represent a two-dimensional projection of the planned path, the combination of two light fans from different directions may support a three-dimensional orientation. Advantageously, the intensity of the light along the intersection line may be higher than in the areas where the light fans do not intersect. This intensity difference may advantageously improve the visibility and/or detectability of the planned path, for example in environments with different light conditions.

Illuminating the planned path with both light fans may include orienting and positioning the light fans so that the light fans project light, for example a predefined light pattern, onto the planned path. The intersection line of the two light fans may advantageously provide precise visual guidance for placing and/or moving the medical object.

The method may advantageously provide full 3D guidance of the medical object, for example 3D needle guidance, for any angulations of the X-ray device, for example a C-arm X-ray device, relative to the planned path, for example a needle path. Furthermore, the method may easily be applied to common clinical workflows. A major clinical advantage is also advantageously to be expected from the method, for example, due to faster and safer needle guidance, less repositioning, a lower radiation dose, and/or a simpler workflow. By combining the current positioning of the first defined arrangement, a minimal number of mechanical degrees of freedom the light guide facilities and use of a projection geometry, guidance of the medical object, for example laser needle guidance, may be realized in the simplest and most economical way possible.

The method may also enable visual guidance for a user for placing and/or moving the medical object along the planned path. Illuminating using the light fans may help the user to detect the three-dimensional orientation and position of the planned path in the space, without X-rays continually having to be taken for this purpose. Improved spatial orientation may be achieved by using two light fans from different directions. Illuminating the planned path using the light fans may thus support precise and low-radiation guidance of the medical object.

A relative positioning of the X-ray source, the X-ray detector, and the two light guide facilities in the first defined arrangement may change, for example, due to mechanical deformations of a common support structure. For example, the relative positioning of the X-ray source, the X-ray detector, and the two light guide facilities may change depending on the current positioning of the first defined arrangement. Advantageously, the change in the relative positioning of the X-ray source, the X-ray detector, and the two light guide facilities may be identified, for example automatically. Identifying the change in the relative positioning of the X-ray source, the X-ray detector, and the two light guide facilities may, for example, include capturing a current relative positioning of the X-ray source, the X-ray detector, and the two light guide facilities, for example, by a sensor. Alternatively or additionally, the change in the relative positioning of the X-ray source, the X-ray detector, and the two light guide facilities may be identified based on a current positioning of the first defined arrangement, for example, based on a look-up table and/or a physical model of the X-ray device.

Advantageously, the emission of the first and/or the second light fan, for example a projection direction and/or a fan angle of the light fan, may be adjusted, for example automatically, depending on the identified change in the relative positioning of the X-ray source, the X-ray detector, and the two light guide facilities. Advantageously, the first and/or second light guide facility may adjust the emission of the respective light fan in such a way, depending on the identified change in the relative positioning, that the respective light fan illuminates the planned path.

A compensation for a change in the relative positioning of the X-ray source, the X-ray detector, and the two light guide facilities may advantageously be enabled as a result when the light fans are emitted.

In another advantageous form of embodiment of the method, the first and/or the second light fan may also be configured as tiltable. Moving the first and/or the second light fan may thereby further include tilting the first and/or second light fan based on the planning information and the current positioning of the first defined arrangement.

Tilting the first and/or second light fan may include changing an orientation and/or inclination of the respective light fan relative to a reference plane and/or reference axis. The tiltability may be realized through mechanical, optical, and/or electro-optical mechanisms. Tilting the first and/or second light fan may, for example, be realized through a tiltable mounting of the respective light guide facility, an adjustable projection mechanism, and/or an adjustable projection matrix. Tilting the respective light fan may advantageously enable a precise orientation of the respective light fan to the planned path.

The first and/or second light fan may each be tiltable around a tilting axis, wherein the tilting axis runs parallel to the layer illuminated by the respective light fan, for example the plane, and wherein at least one, virtual or real, fan beam of the respective light fan has an angle with respect to the tilting axis of between 45 and 90 degrees. The respective tilting axis of the first and/or second light fan may advantageously be arranged vertically to the rotation axis of the respective light fan.

Tilting the first and/or second light fan based on the planning information and the current positioning may include adjusting the orientation and/or inclination of the first and/or second light fan to the spatial positioning of the planned path and the current spatial positioning of the first defined arrangement, for example in such a way that the first and the second light fan illuminate the planned path. The first and/or second light fan may be tilted in addition to being rotated around the respective rotation axis.

The first and/or second light fan may, for example, be tilted in an angular range of −45° to +45° relative to an initial positioning of the respective light fan. The first and/or second light fan may be tilted incrementally or continuously. When tilting incrementally, predefined angular positions may, for example, be used. When tilting continuously, a tilting angle may be adjusted in a stepless manner.

The control unit of the X-ray device may be configured to control the tilting of the first and/or second light fan. The planning information and the current positioning of the first defined arrangement may be used by the control unit to calculate an optimal tilting angle for the respective light fan. How the planned path is arranged relative to the current or a future, for example planned, position of the first defined arrangement may thereby be taken into account.

Greater flexibility may be achieved when illuminating the planned path due to the additional tiltability of the first and/or second light fan. This may be particularly advantageous when the planned path runs at a complex angle to the first defined arrangement and/or when the planned path runs outside a predefined distance range with respect to the first defined arrangement, for example if it falls short of a predefined minimum distance or exceeds a predefined maximum distance. The combination of rotating and tilting the first and/or second light fan may enable a precise three-dimensional orientation of the respective light fan to the planned path. For example, this may enable more precise illumination of the planned path, for example when the path does not run parallel to the rotation axis of the respective light fan.

Advantageously, more precise needle guidance may be enabled even in difficult anatomical conditions due to the additional tiltability of the first and/or second light fan. The increased flexibility when orienting the light fans may lead to improved visualization of the planned path and thus increase precision and safety during medical procedures.

In addition to the rotation, the second light fan may also be freely tiltable in all spatial directions, for example it may be tilted. This may enable even more flexible adjustment of the second light fan to various positions and orientations of the planned path.

In another embodiment of the method, a focusing of the first and/or the second light fan may be adjusted in such a way, based on the planning information and a current positioning of the first defined arrangement, that the respective light fan has a predefined geometry in an area of the planned path.

Adjusting the focusing of the first and/or the second light fan may include adjusting optical properties of the respective light fan. Adjusting the focusing of the first and/or the second light fan may, for example, include changing a width, for example a line width of a projected line, sharpness, and/or intensity of the respective light fan in a certain area. The focusing of the first and/or the second light fan may be adjusted using a variety of technical mechanisms. For example, the focusing, for example a focus, of the first and/or second light fan may be adjusted by adjustable optics. Alternatively or additionally, the focusing of the first and/or second light fan may be adjusted by adaptive optics, for example dynamically.

Adjusting the focusing of the first and/or the second light fan may include adjusting focusing optics of the respective light guide facility. The focusing optics may thereby be set so that the respective light fan has a suitable width, layer thickness, and/or path display accuracy in the area of the planned path for the required accuracy of the needle guidance task. Adjusting the focusing of the first and/or second light fan may, for example, include motorized adjustment of optical elements, for example, optical lenses and/or mirrors in the respective light guide facility.

The planning information may also have information about a predefined geometry of the first and/or second light fan in the area of the planned path. This information may, for example, include a predefined width, sharpness, and/or intensity of the first and/or second light fan at predefined points along the planned path.

The area of the planned path may refer to a certain spatial section and/or a certain region along the planned path. This area may, for example, be defined by a distance between the intersection point of the planned path and the rotation axis of the first light fan.

The predefined geometry of the first and/or second light fan may include a certain form, width, and/or intensity distribution of the first and/or second light fan. The predefined geometry may be specified in such a way that it enables improved visibility, visualization, and/or guidance for the medical object along the planned path.

The focusing of the first and/or second light fan may be adjusted, for example automatically, by a control unit of the X-ray device. The control unit may be configured to process the planning information and the current positioning and to send corresponding control signals to the respective light guide facility, in order to adjust the focusing of the respective light fan.

Adjusting the focusing of the first and/or second light fan based on the planning information may, for example, include taking into account a length of the planned path, its orientation in the space, and/or a type of the medical object to be guided. The current positioning of the first defined arrangement may also be incorporated when adjusting the focusing of the first and/or second light fan, in order to take into account a spacing between the respective light guide facility and the planned path, for example.

By adjusting the focusing of the first and/or second light fan, improved visualization and guidance of the medical object along the planned path may be achieved. This may lead to improved accuracy when placing the medical object and thus improve the safety and efficiency of medical procedures. Additionally, flexible adjustment of the light fans may be enabled for different clinical scenarios and requirements, which may for example increase the versatility of the X-ray device.

In another embodiment of the method, the rotation axis of the first light fan may be arranged within the layer that is illuminated by the first light fan. Alternatively or additionally, the rotation axis of the second light fan may be arranged within the layer that is illuminated by the second light fan.

A layer may be defined as a thin, flat structure or plane. The respective layer that is illuminated by the first or second light fan may include a spatial area that is entered by the light of the respective light fan.

The respective rotation axis of the first and/or second light fan may be arranged within the layer illuminated by the respective light fan when the rotation axis primarily runs within the spatial area illuminated by the respective light fan.

Arranging the rotation axis of the first light fan within the layer illuminated by the first light fan may be achieved by mounting the first light guide facility in a suitable, for example rotatable, manner. For example, the first light guide facility may be configured in such a way that the first light fan originates on the rotation axis of the first light fan. This may be achieved through precise orientation of the light source and/or the optical components within the first light guide facility.

Similarly, the rotation axis of the second light fan may be arranged within the layer illuminated by the second light fan by mounting the second light guide facility in a corresponding, for example rotatable, manner. The second light guide facility may be configured in such a way that the second light fan originates on the rotation axis of the second light fan. This may be achieved through precise orientation of the light source and/or the optical components within the second light guide facility.

Advantageously, arranging the rotation axis of the first light fan within the layer illuminated by the first light fan may enable that the planned path may be illuminated by a rotation of the first light fan around its rotation axis after the first defined arrangement has been positioned. Furthermore, arranging the rotation axis of the second light fan within the layer illuminated by the second light fan may advantageously simplify moving the second light fan to illuminate the planned path, for example as the rotation axes of the first and second light fan intersect.

In another embodiment of the method, repositioning the first defined arrangement into another positioning may be restricted to a rotation around an intersection point of the rotation axis of the first light fan with the planned path, a combination including a rotation around the intersection point and a translation parallel to the planned path, or a combination including a rotation around the intersection point and a translation parallel to the rotation axis of the first light fan. The other positioning may be specified as the current positioning of the first defined arrangement. The first and/or the second light fan may be moved in such a way, based on the planning information and a current positioning of the first defined arrangement, that the first and the second light fan illuminate the planned path. Moving the first and the second light fan may thereby include at least one rotation of the first and/or the second light fan around the respective rotation axis.

Specifying the other positioning as the current positioning may include updating the current positioning with the other positioning. The other positioning may thereby have been captured in advance. The other positioning of the first defined arrangement may be captured analogous to the capture of the current positioning of the first defined arrangement.

Repositioning may include a new positioning of the first defined arrangement. The first defined arrangement may thereby be moved into another spatial position, orientation, and/or pose, which may be referred to as the other positioning.

The repositioning of the first defined arrangement may be controlled by a control unit of the X-ray device. The control unit may be configured to restrict the movements of the first defined arrangement to the named combinations. This may enable precise and controlled positioning of the X-ray device in relation to the planned path.

According to a first variant, repositioning the first defined arrangement into the other positioning may be restricted to a rotation around the intersection point of the rotation axis of the first light fan with the planned path. An intersection point may be defined as a point at which two or more lines, areas, or objects cross or meet. In this instance, the intersection point may be the point at which the rotation axis of the first light fan intersects the planned path. A rotation may include a rotational movement around an axis or a rotation point. In this context, the rotation of the first defined arrangement may be effected around the intersection point of the rotation axis of the first light fan with the planned path, wherein the intersection point serves as the rotation point. For example, the rotation of the first defined arrangement around the intersection point of the rotation axis of the first light fan with the planned path may enable an isocentric rotation of the first defined arrangement around the intersection point. This may be advantageous for obtaining various views of the planned path, while the intersection point is maintained as the reference point.

According to another variant, repositioning the first defined arrangement into the other positioning may be restricted to a combination including a rotation around the intersection point and a translation parallel to the planned path. The translation parallel to the planned path may enable the movement of the first defined arrangement along the planned path. The intersection point of the rotation axis of the first light fan with the planned path may thereby be moved with it along the planned path. Combining the rotation around the intersection point and the translation parallel to the planned path may include a composite, for example temporally successive and/or interleaved, and/or simultaneous rotation around the intersection point and translation parallel to the planned path. This may be particularly useful with extended planned paths or when various areas along the path are of interest. Additionally, this embodiment may be advantageous when another area of the examination object needs to be imaged using the X-ray device.

According to another variant, repositioning the first defined arrangement into the other positioning may be restricted to a combination including a rotation around the intersection point and a translation parallel to the rotation axis of the first light fan. The translation of the first defined arrangement parallel to the rotation axis of the first light fan may enable adjusting a spacing between the first defined arrangement and the planned path. Combining the rotation around the intersection point and the translation parallel to the rotation axis of the first light fan may include a composite, for example temporally successive and/or temporally interleaved, and/or simultaneous rotation around the intersection point and translation parallel to the rotation axis of the first light fan. This may be advantageous for finding the improved imaging position and/or for creating space for other medical instruments and/or facilities.

Combining the respective rotation and translation of the first defined arrangement may enable a more complex movement, including both a rotation and a straight-line translatory movement. This combined movement may enable more precise positioning of the first defined arrangement.

Advantageously, this embodiment of the method may enable precise and flexible control of the X-ray device, allowing the planned path to be illuminated from various angles and positions, while a consistent reference is simultaneously maintained. This may improve the accuracy and efficiency of medical procedures requiring precise guidance of medical objects, for example instruments, along a planned path.

In another embodiment of the method, the rotation axis of the first light fan may run through a rotation center, for example an isocenter, of the first defined arrangement, a geometric center of the X-ray detector, the X-ray source, and/or a geometric center of a radiation exit window of the X-ray source.

The rotation center of the first defined arrangement may be defined as a point in the space, around which the first defined arrangement is rotatably mounted. The rotation center may, for example, be configured as an isocenter of the first defined arrangement. The isocenter may be defined as a point in the space, which remains fixed during all rotations of the first defined arrangement. This may be particularly advantageous with an arrangement of the second light guide facility in the second defined arrangement with the X-ray detector. A planned path of any orientation, for example a needle path, running through the isocenter of the first defined arrangement, may thereby be illuminated for three-dimensional light guidance (3D light guidance) by the crossed light fans by rotating the X-ray detector, for example the second defined arrangement, and rotating the second light fan.

A user, for example, a doctor, may also rotate the first defined arrangement isocentrically at will, for example, by a joystick and/or automatic isocentric rotations between saved angulations, wherein the two light fans, for example the laser needle guidance, continually illuminate the planned path with full 3D information. This may enable very flexible use of the laser needle guidance in the clinical workflow. An isocentric rotation is quick and easy to perform mechanically with standard arm X-ray devices, for example, a C-arm X-ray device or an O-arm X-ray device. The arm X-ray device may thereby also be floor-and/or ceiling-supported.

The geometric center of the X-ray detector may be defined as a point on a detector surface, for example the X-ray sensitive layer of the X-ray detector, which has an even spacing to all edges and/or corners of the X-ray detector. With a rectangular X-ray detector, the geometric center may, for example, be configured as the intersection point of diagonals of the rectangle.

The radiation exit window of the X-ray source may be defined as an area of the X-ray source, through which the produced X-rays are issued. The radiation exit window may, for example, be configured as an opening in a screen of the X-ray source. The geometric center of the radiation exit window of the X-ray source may be defined as a point on the surface of the radiation exit window, which has an even spacing to all edges and/or corners of the radiation exit window.

The rotation axis of the first light fan may run through one or more of the points or components named above. For example, the rotation axis of the first light fan may run through the rotation center of the first defined arrangement and the geometric center of the X-ray detector. Alternatively, the rotation axis of the first light fan may run through the X-ray source and the geometric center of the radiation exit window of the X-ray source. This may include the rotation axis of the first light fan intersecting these points or passing by them very closely.

This arrangement of the rotation axis of the first light fan may offer multiple technical advantages. On the one hand, precise orientation of the first light fan may be achieved relative to the most important components of the X-ray device. This may improve the accuracy of the needle guidance. On the other hand, this arrangement may simplify the calibration and maintenance of the system, as the rotation axis of the first light fan correlates with easily identifiable points of the X-ray device. Moreover, this configuration may simplify integration of the light guidance system into existing X-ray devices, as it is guided by the underlying geometric properties of the X-ray device.

In another embodiment of the method, the medical object may be arranged along the planned path. X-rays may thereby be emitted by the X-ray source to illuminate the medical object. In another step, the X-rays may be captured by the X-ray detector and a signal may be provided depending on the captured X-rays. In another step, X-ray image data may be provided depending on the signal.

The medical object, for example a longitudinal extension axis of the medical object, may advantageously be arranged along the planned path, for example on the planned path. The medical object may advantageously have been arranged along the planned path before beginning the method.

The emission of X-rays to illuminate the medical object may include an emission of X-ray photons by the X-ray source. The emission of X-rays may be controlled by a control unit of the X-ray device. The X-rays may be oriented so that they penetrate the medical object and make contact with the X-ray detector.

Capturing the X-rays by the X-ray detector may include a detection and conversion of the incidental X-ray photons into electrical signals. The X-rays may be captured, for example detected, by the X-ray detector, for example the X-ray sensitive layer of the X-ray detector, for example after interacting with the medical object.

The X-ray detector may be configured to convert the impinging X-rays into electrical and/or digital signals. The X-ray detector may provide a signal depending on the captured X-rays. This signal may be provided to the control unit of the X-ray device. Providing the signal depending on the captured X-rays may include producing and transmitting electrical and/or digital signals that have information about the detected X-rays. These signals may, for example, represent intensity values for different detector areas.

Providing X-ray image data depending on the signal may include processing and preparing the signals provided by the X-ray detector into digital image data, for example reconstructing the X-ray image data based on the signal. The X-ray image data may include a representation, for example an image, of the medical object, for example the medical object and the examination object. The X-ray image data may thereby be two-dimensionally (2D) and/or three-dimensionally (3D) spatially resolved. The X-ray image data may also be temporally resolved. The X-ray image data may have multiple image points, for example pixels and/or voxels, each with at least one image value, for example multiple image values, for example, time-intensity curves, which each image a partial volume. The X-ray image data may be provided depending on the signal. The X-ray image data may be provided by the control unit. Providing the X-ray image data may include storing on a computer-readable storage medium, and/or displaying on a display unit, and/or transferring to a provision unit. For example, a graphic representation of the X-ray image data may be displayed by the display unit.

The X-ray image data may have information about an, for example current, position and orientation of the medical object along the planned path. This information may be used to review the positioning of the medical object and correct it as necessary.

Combining laser needle guidance by the light fans and X-ray imaging may offer multiple advantages. On the one hand, the laser needle guidance may enable precise orientation of the medical object along the planned path, without X-rays continually having to be emitted. On the other hand, the X-ray image data may enable a review of the actual position of the medical object with respect to the examination object. This may reduce the radiation exposure for the examination object and medical personnel, as fewer X-rays may be required for position control. Additionally, combining the two methods may increase accuracy and safety when positioning the medical object.

In another embodiment of the method, the planning information may be registered with a coordinate system of the X-ray device.

The coordinate system of the X-ray device may, for example, be a three-dimensional Cartesian coordinate system, which originates at a defined point of the X-ray device. This defined point may, for example, be the isocenter of the X-ray device, the center of the X-ray detector, or another suitable reference point.

Registering the planning information with the coordinate system of the X-ray device may include a spatial assignment of the data about the coordinate system of the X-ray device that is included in the planning information. Registering the planning information with the coordinate system of the X-ray device may also include a transformation of the planning information in the coordinate system of the X-ray device. This may, for example, include a coordinate transformation, a rotation, scaling, deformation, and/or a translation of the planning information.

The registration may be performed in different ways, for example: by using markers and/or reference points, which are known in both the planning information and in the coordinate system of the X-ray device, by registering images, in which the image data is compared to image data of the X-ray device, for example, anatomical landmarks of the examination object, by a user manually inputting registration points, by using an external tracking system, which captures both the current position of the examination object and the current position of the X-ray device, and/or by providing the planning information, for example planning of the planned path, based on image data that has been recorded by the X-ray device, for example immediately prior.

Registration may be used to set the information contained in the planning information, for example, the planned path, in relation to the current positioning of the X-ray device, for example of the first defined arrangement. This may enable precise orientation of the light fans and precise positioning of the X-ray device.

The control unit of the X-ray device may register the planning information with the coordinate system of the X-ray device. The control unit may be configured to carry out the necessary calculations and transformations.

Registering the planning information with the coordinate system of the X-ray device enables precise orientation of the light fans on the planned path. This may lead to improved accuracy when positioning the medical object.

Advantageously, seamless integration of pre-operative planning and intra-operative support may be achieved by registering the planning information with the coordinate system of the X-ray device. This may increase the efficiency and safety of the medical procedure and simplify workflows for the medical personnel.

Embodiments relate in a second aspect to an X-ray device, including an X-ray source and an X-ray detector opposite each other and two light guide facilities arranged spaced apart from each other. The X-ray source, the X-ray detector, and the light guide facilities are arranged in a first defined arrangement. The first defined arrangement is movably mounted. A first light guide facility of the two light guide facilities is configured to emit a first light fan and a second light guide facility of the two light guide facilities is configured to emit a second light fan. The first and the second light fan are at least rotatable around one rotation axis in each instance. The rotation axis of the first light fan intersects a detector surface of the X-ray detector and the rotation axis of the second light fan in an operating state of the X-ray device. The X-ray device, for example its components, is also configured to execute a method for controlling an X-ray device.

The advantages of the X-ray device essentially correspond to the advantages of the method for controlling an X-ray device. The features, advantages, or alternative forms of embodiment mentioned here may also be transferred to the other claimed subject matters and vice versa.

Advantageously, the X-ray device may also include a control unit. The control unit may be configured, in the operating state: to receive planning information for a planned path for arranging a medical object, which may be imaged for example by the medical X-ray device; to position the first defined arrangement in such a way that the rotation axis of the first light fan intersects the planned path; to move the first and/or the second light fan in such a way, based on the planning information and the current positioning of the first defined arrangement, that the first and the second light fan illuminate the planned path; wherein moving the first and/or the second light fan includes at least one rotation of the first and/or the second light fan around the respective rotation axis.

For example, the X-ray device may advantageously constitute a solution that may be realized at lower cost, with comparatively little installation space, and with reduced complexity compared to a solution with two laser modules on the X-ray detector that are fully tiltable and movable, for example freely, in all spatial axes.

The control unit may advantageously include an interface, a computing unit, and/or a memory unit. The interface to the control unit may be configured to receive planning information and to send control signals to the light guide facilities and other components of the X-ray device. The control unit may be configured to control the positioning of the first defined arrangement and the movement of the first and/or second light fan by providing a respective control signal by the interface.

The computing unit of the control unit may be configured to process the received planning information and to perform calculations for the positioning of the first defined arrangement and the movement of the light fans. It may execute algorithms to determine an optimal orientation of the light fans based on the planned path and the current positioning.

The memory unit of the control unit may be configured to store the planning information, interim results of the calculations, and/or configuration data of the X-ray device. The memory unit may also include pre-programmed movement sequences and calibration data for the light guide facilities.

The control unit may also include software modules that implement the specific functions, such as registering the planning information with the coordinate system of the X-ray device, capturing the current positioning, and/or controlling the rotational movements of the light fans.

A real-time operating system of the control unit may ensure time-critical coordination of the different method steps, in order to enable precise and synchronized execution of the light fan movements with the positioning of the X-ray device.

The components of the control unit may be connected to each other via an internal data bus, in order to enable fast data exchange and efficient processing. This may enable seamless integration of the different method steps and rapid adjustment of the light fan orientation to changes in the positioning or the planned path.

In another embodiment of the X-ray device, the first and second light fan may each be rotatable by a mounting, which may be rotated, for example in a motorized manner, of the respective light guide facility, and/or a projection mechanism that may be adjusted, for example in a motorized manner, and/or an adjustable projection matrix.

The rotatable mounting of the respective light guide facility may be configured as a mechanical apparatus that enables a rotational movement of the light guide facility around a defined axis, for example the rotation axis of the respective light fan. The rotatable mounting may, for example, be executed as ball bearings, slide bearings, and/or roller bearings. A motorized execution of the rotatable mounting may include an electric motor that drives the rotational movement of the light guide facility. The electric motor may, for example, be configured as a stepper motor and/or servo motor. A motorized execution may thereby enable precise and automated control of the rotation.

The adjustable projection mechanism may be configured as a mechanical and/or optomechanical apparatus that enables a change in the projection direction of the respective light fan. The adjustable projection mechanism may, for example, include a moving mirror, a prism, and/or a lens, the position and/or orientation of which may be changed to adjust the direction of the respective light fan. The projection mechanism may also be driven by a motor, in order to ensure precise positioning.

An adjustable projection matrix may be configured as an electronic and/or optoelectronic apparatus that enables a change in the projection direction and/or form of the light fan. The adjustable projection matrix may, for example, be executed as a digital micromirror (DMD), liquid crystal light modulator (LCoS), and/or as a matrix of individually controllable light sources. The adjustable projection matrix may be configured as an arrangement of individually controllable light elements that enable dynamic adjustment of the form and/or direction of the respective light fan, without mechanical movements having to be performed.

Rotating the first and/or the second light fan may be realized through a combination of the rotatable mounting, the adjustable projection mechanism, and/or the adjustable projection matrix. For example, the rotatable mounting may enable a rough orientation of the light guide facility, while the adjustable projection mechanism and/or projection matrix enable fine tuning of the light guide facility.

The motorized execution of the rotatable mounting, the adjustable projection mechanism, and/or the adjustable projection matrix may enable precise and automated control of the light fan orientation. Advantageously, a respective plane and/or layer of the first and/or second light fan may be changed, for example may be moved, through at least one motorized movement axis relative to the X-ray detector. Other types of variable optical images are also conceivable for changing the respective light fan planes.

The control unit may be configured to control the mounting, which may be rotated, for example in a motorized manner, of the respective light guide facility, and/or the projection mechanism that may be adjusted, for example in a motorized manner, and/or the adjustable projection matrix.

Advantageously, the embodiment of the X-ray device may enable flexible and precise needle guidance. The various options for adjusting and positioning the light fans may enable an improved configuration for different clinical requirements and patient anatomies. The possibility of controlling the light fans independently of each other may enable improved 3D visualization of the planned path, thus increasing the accuracy and safety of needle placement.

In another embodiment of the X-ray device, the first and/or the second light fan may each also be tiltable, for example may be tiltably mounted, by a mounting, which may be tilted, for example in a motorized manner, of the respective light guide facility, and/or a projection mechanism that may be adjusted, for example in a motorized manner, and/or an adjustable projection matrix.

The tiltable mounting of the first and/or the second light fan may be configured as an option for changing an inclination and/or orientation of the respective light fan relative to a reference plane. The tiltable mounting may be realized through mechanical joints, ball joints, and/or other apparatuses, which enable a rotation around at least one axis that does not correspond to the rotation axis of the respective light fan.

A mounting that may be tilted in a motorized manner may be configured as an apparatus that enables changing the inclination of the respective light fan automatically by a motor and/or actuator. The mounting that may be tilted in a motorized manner may, for example, include stepper motors, servo motors, and/or linear motors, which enable precise movements and may be controlled by the control unit of the X-ray device.

An adjustable projection mechanism may be configured as an apparatus that enables changing the projection of the respective light fan. The adjustable projection mechanism may, for example, include a moving mirror, an adjustable lens system, and/or an electronically controllable optical element. By adjusting the projection mechanism, the orientation, form, and/or size of the respective light fan may be adjusted.

An adjustable projection matrix may be configured as an arrangement of optical elements that may be controlled individually, in order to influence the projection of the light fan. The adjustable projection matrix may, for example, include a plurality of micromirrors, liquid crystal cells, and/or other controllable optical elements. The projection of the light fan may be precisely adjusted by selectively controlling these elements.

The tiltable mounting of the respective light guide facility may offer a mechanical basis for adjusting the inclination of the light fan.

The adjustable projection mechanism may enable the tiltability of the respective light fan through optomechanical mechanisms. For example, a moving mirror and/or an adjustable prism system may be used to change the direction of the respective light fan, without the entire light guide facility having to be moved. This may enable finer and faster adjustment of the respective light fan orientation.

The adjustable projection matrix may constitute an electronic method for changing the respective light fan orientation. By selectively activating or deactivating individual elements of the projection matrix, the form and/or direction of the respective light fan may be adjusted. This may enable very precise and flexible control of the respective light fan orientation without moving parts.

In combination, these technologies may enable a multi-stage adjustment of the light fan orientation. The tiltable mounting of the respective light guide facility may be used for rough adjustments, while the projection mechanism and/or the projection matrix enable finer tuning. This may enable improved accuracy and flexibility when orienting the respective light fan to the planned path.

The motorized execution of these components may enable automated and precise control of the tilting movements.

By combining these technologies, the X-ray device may be configured to orient the light fans across a wide range of angles and with high precision. This may improve the ability of the X-ray device to navigate complex anatomical structures and may enable precise needle guidance in various clinical scenarios.

In another embodiment of the X-ray device, the first light guide facility may be arranged in a second defined arrangement with respect to the X-ray detector. The second defined arrangement may be rotatably mounted. The rotation axis of the first light fan may correspond to a rotation axis of the second defined arrangement.

The first light guide facility may be arranged in a second defined arrangement with respect to the X-ray detector. The second defined arrangement may describe a defined, for example rigid, spatial relationship between the first light guide facility and the X-ray detector, for example a detector housing of the X-ray detector. This arrangement may, for example, include a certain spatial position, orientation, and/or pose of the first light guide facility relative to the X-ray detector.

The second defined arrangement may be rotatably mounted. This means that the first light guide facility may be rotated together with the X-ray detector around a rotation axis. The rotatable mounting may, for example, be realized by a pivot bearing, a ball bearing, and/or another suitable rotation element. For example, the X-ray detector may be rotatable by a motor. The first light guide facility may also be rotated by rotating the X-ray detector.

The rotation axis of the first light fan may correspond to a rotation axis of the second defined arrangement. This means the rotation axis around which the first light fan is rotatably mounted may correspond to the rotation axis around which the second defined arrangement is rotatably mounted. This may enable precise orientation of the first light fan, as the rotation of the light fan is directly coupled to the rotation of the second defined arrangement. As a result, the first light fan may, for example, be rotated with a specified positioning, for example angulation, of the X-ray device, for example of the first defined arrangement and/or of a C-arm of the X-ray device, by rotating the X-ray device.

For example, the first and second light guide facility may be arranged at different edges and/or corners of the X-ray detector.

This may enable a simple and reliable method for orienting the first light fan, as no separate rotation mechanism is required for the first light guide facility.

In another embodiment of the X-ray device, the first and/or the second light guide facility may each be arranged on the X-ray detector, the X-ray source, or a guide unit.

The guide unit may be configured as a mechanical structure that is configured to hold and/or position the components of the X-ray device. The guide unit may, for example, be configured as a C-arc, for example a C-arm, O-arm, bracket, stand, and/or other suitable support structure. The guide unit on which a light guide facility may be arranged, may, for example, include a separate arm and/or bracket that may be positioned independently of the X-ray source and X-ray detector. This may provide additional flexibility when orienting the light fans.

Arranging the first and/or second light guide facility on the X-ray detector, X-ray source, or guide unit may include a fixed or detachable connection. A fixed connection may, for example, be realized using screws, adhesive, and/or welding. A detachable connection may, for example, be realized using clamps, magnets, and/or quick locks. Alternatively or additionally, the first and/or the second light guide facility may each be integrated at least partially, for example fully, into the X-ray detector, the X-ray source, or the guide unit.

The arrangement of the light guide facilities may be selected so that improved illumination of the planned path is enabled. The position of the light guide facilities may thereby be adjusted to the specific requirements of the respective medical application.

The arrangement of the light guide facilities on different components of the X-ray device may be advantageous, in order to achieve improved coverage of a working area. For example, one light guide facility may be arranged on the X-ray detector and another light guide facility may be arranged on the X-ray source, in order to emit the light fans from different directions.

Advantageously, the option of arranging and orienting the light guide facilities flexibly may enable precise three-dimensional light guidance for various medical procedures, while simultaneously enabling X-ray images to be taken from different angles. This may improve the accuracy and efficiency of medical procedures and simultaneously reduce the radiation exposure for the examination object.

The plane of the second light fan may be changed relative to the X-ray detector by at least one motorized movement axis. The respective light fan plane may, for example, be changed by moving and/or rotating the respective light guide facility and/or using a moving mirror mechanism.

At least one of the light guide facilities, for example, the second light guide facility, may be arranged in a center of the C-arm.

In another embodiment of the X-ray device, the first defined arrangement may be mounted translatably and/or rotatably.

Translating the first defined arrangement may include moving the first defined arrangement in one or more spatial directions. The first defined arrangement may, for example, be movable along a horizontal and/or vertical axis. For example, the first defined arrangement may be translatable manually or using a motor.

Rotating the first defined arrangement may include turning the first defined arrangement around one or more rotation axes. The first defined arrangement may, for example, be rotatable around a vertical, horizontal, and/or diagonal axis. For example, the first defined arrangement may be rotatable manually or using a motor.

The translatable and/or rotatable mounting of the first defined arrangement may enable flexible positioning of the X-ray device. As a result, the X-ray device may be adjusted for various examination situations and examination object positionings.

According to another embodiment of the X-ray device, the X-ray source may be configured to emit X-rays to illuminate the medical object. Furthermore, the X-ray detector may be configured to capture the X-rays and to provide a signal to the control unit depending on the captured X-rays. Moreover, the control unit may be configured to provide X-ray image data depending on the signal.

Embodiments relate in a third aspect to a computer program product with a computer program, that may be loaded directly into a memory of a control unit. The computer program product includes program sections, in order to execute all steps of a method for controlling an X-ray device, if the program sections are executed by the control unit.

The computer program product may include software with a source code that still needs to be compiled and bound or that only needs to be interpreted, or an executable software code that only needs to be loaded into the control unit for execution. A control unit may execute the method for controlling an X-ray device in a rapid, robust and identically repeatable manner by the computer program product. The computer program product may be configured so that it may execute the method steps by the control unit.

The computer program product may, for example, be saved to a computer-readable storage medium or be stored on a network or server, from where it may be loaded onto the processor of a control unit, which may be directly linked to the control unit or be configured as part of the control unit. Control information of the computer program product may also be stored on an electronically readable data carrier. The control information of the electronically readable data carrier may be arranged in such a way that it executes a method when the data carrier is used in a control unit. Examples of electronically readable data carriers may be a DVD, a magnetic tape, or a USB stick, on which electronically readable control information, for example software, may be stored. If this control information is read from the data carrier and stored in a control unit, all forms of embodiment of the methods described above may be executed.

An advantage of largely software-based implementation may be that previously used control units may also be easily retrofitted through a software update. In addition to the computer program, such a computer program product may include, where necessary, additional elements such as documentation and/or additional components, as well as hardware components such as hardware keys (dongles etc.) for using the software.

Program sections may be parts or modules of the computer program that execute specific functions or tasks. The program sections may be written in various programming languages and may include instructions, functions, classes, and/or objects, which may be executed or processed by the control unit.

### BRIEF DESCRIPTION OF THE FIGURES

FIG. 1 depicts a schematic representation of a method for controlling an X-ray device according to an embodiment.

FIG. 2 depicts a schematic representation of a method for controlling an X-ray device with focusing adjustment according to an embodiment.

FIG. 3 depicts a schematic representation of a method for controlling an X-ray device with registration of planning information according to an embodiment.

FIG. 4 depicts a schematic representation of a method for controlling an X-ray device with provision of X-ray image data according to an embodiment.

FIGS. 5 to 8 show schematic representations of various embodiments of a X-ray device.

FIGS. 9 and 10 show schematic representations of various positionings of a second defined arrangement according to an embodiment.

FIG. 11 depicts a schematic representation of a X-ray device according to an embodiment.

### DETAILED DESCRIPTION

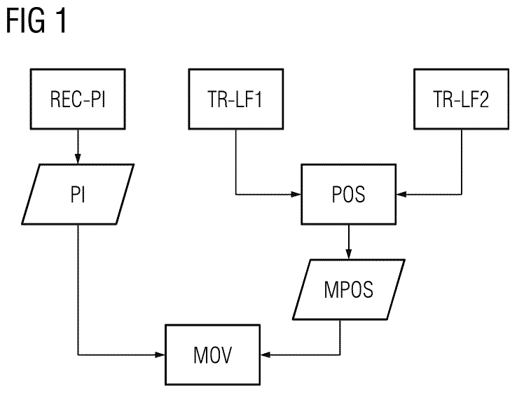

FIG. 1 depicts a schematic representation of an advantageous form of embodiment of a method for controlling an X-ray device. The X-ray device may include an X-ray source and an X-ray detector opposite each other and two light guide facilities arranged spaced apart from each other, which may be arranged in a first defined arrangement. The first defined arrangement may be movably mounted. A first light guide facility of the two light guide facilities may be configured to emit a first light fan and a second light guide facility of the two light guide facilities may be configured to emit a second light fan. The first and the second light fan may be at least rotatable around one rotation axis in each instance. The rotation axis of the first light fan may intersect the rotation axis of the second light fan and a detector surface of the X-ray detector. In a first step, planning information PI may be received REC-PI for a planned path for arranging a medical object MO, which may be imaged for example by the medical X-ray device. In another step, the first light fan may be emitted TR-LF1 by the first light guide facility. In another step, the second light fan may be emitted TR-LF2 by the second light guide facility. In another step, the first defined arrangement may be positioned POS in such a way that the rotation axis of the first light fan intersects the planned path. In another step, the first and/or the second light fan may be moved MOV in such a way based on the planning information PI and a current positioning MPOS of the first defined arrangement that the first and the second light fan illuminate the planned path. Moving MOV the first and/or the second light fan may include at least one rotation of the first and/or the second light fan around the respective rotation axis.

In another embodiment, the first and/or the second light fan may also be configured as tiltable. Moving MOV the first and/or the second light fan may further include a tilting of the first and/or second light fan based on the planning information PI and the current positioning MPOS of the first defined arrangement.

Repositioning the first defined arrangement into another positioning may be restricted to a rotation around an intersection point of the rotation axis of the first light fan with the planned path, a combination including a rotation around the intersection point and a translation parallel to the planned path, or a combination including a rotation around the intersection point and a translation parallel to the rotation axis of the first light fan. The other positioning may be specified as the current positioning MPOS of the first defined arrangement. The first and/or the second light fan may be moved MOV in such a way, based on the planning information PI and a current positioning MPOS of the first defined arrangement, that the first and the second light fan illuminate the planned path. Moving MOV the first and the second light fan may include at least one rotation of the first and/or the second light fan around the respective rotation axis.

The rotation axis of the first light fan may run through a rotation center, for example an isocenter, of the first defined arrangement, a geometric center of the X-ray detector, the X-ray source, and/or a geometric center of a radiation exit window of the X-ray source.

The rotation axis of the first light fan may also be arranged within the layer that is illuminated by the first light fan. Alternatively or additionally, the rotation axis of the second light fan may be arranged within the layer that is illuminated by the second light fan.

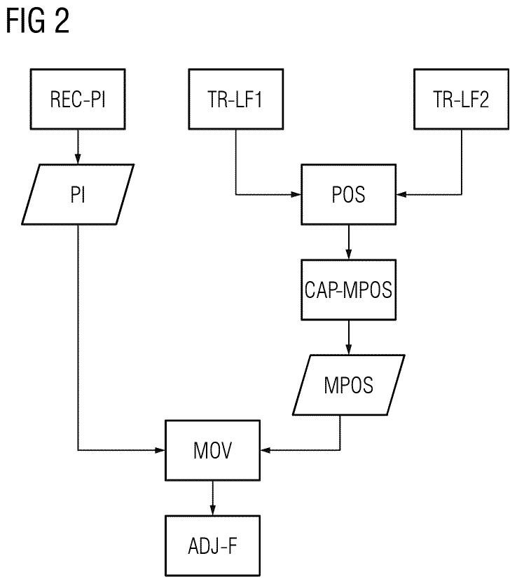

FIG. 2 depicts a schematic representation of another embodiment of a method for controlling an X-ray device. The method may thereby further include adjusting the focusing ADJ-F of the first and/or second light fan. The focusing of the first and/or the second light fan may be adjusted ADJ-F in such a way, based on the planning information PI and a current positioning MPOS of the first defined arrangement, that the respective light fan has a predefined geometry in an area of the planned path.

Moreover, the method may include capturing CAP-MPOS the current positioning of the first defined arrangement.

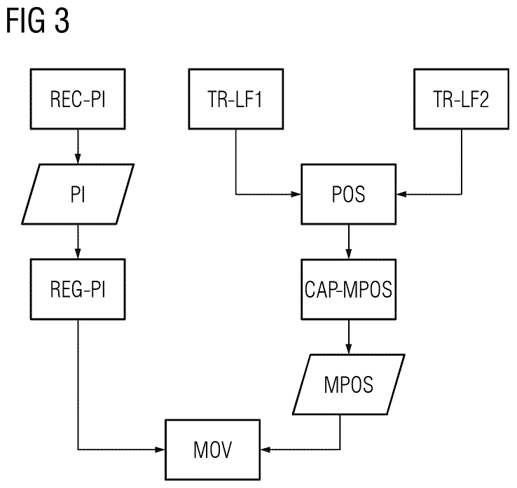

FIG. 3 depicts a schematic representation of another embodiment of a method for controlling an X-ray device. The method may thereby further include registering REG-PI the planning information PI with a coordinate system of the X-ray device. The planned path, for example a planned needle path, may hereby be present in a coordinate system registered to the X-ray device.

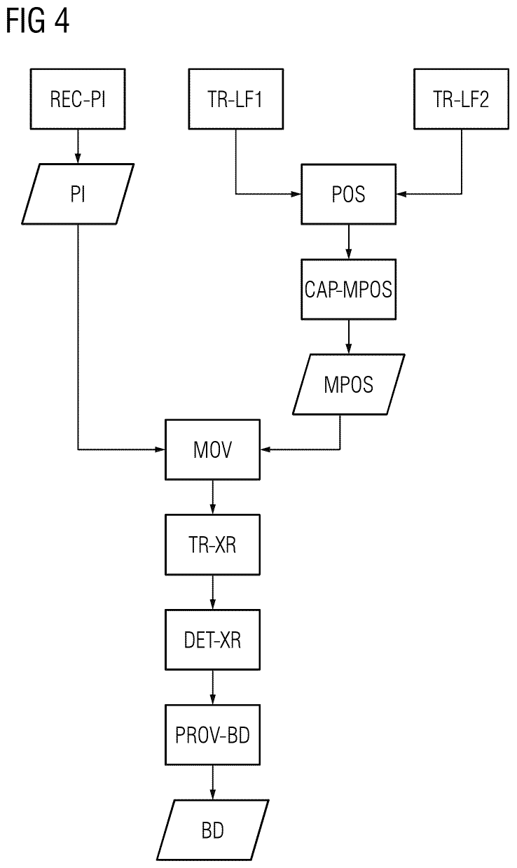

FIG. 4 depicts a schematic representation of another embodiment of a method for controlling an X-ray device. X-rays may thereby be emitted TR-XR by the X-ray source to illuminate the medical object MO. The X-rays may also be captured DET-XR by the X-ray detector, wherein the X-ray detector may provide a signal depending on the captured X-rays. X-ray image data BD may be provided PROV-BD on the basis of the provided signal.

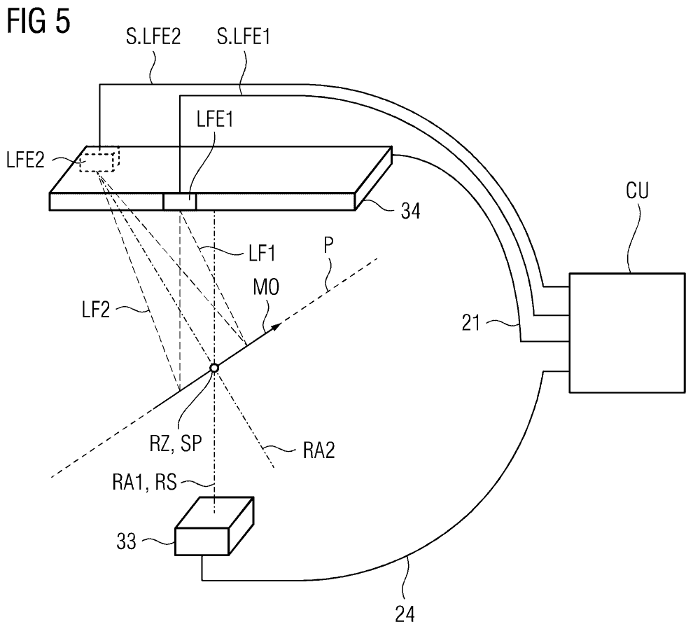

FIG. 5 depicts a schematic representation of an embodiment of a X-ray device. The X-ray device may include an X-ray source 33 and an X-ray detector 34 opposite each other and two light guide facilities LFE1, LFE2 arranged spaced apart from each other. The X-ray source 33, the X-ray detector 34, and the light guide facilities LFE1, LFE2 may be arranged in a first defined arrangement. The first defined arrangement may be movably mounted. A first light guide facility LFE1 of the two light guide facilities may be configured to emit a first light fan LF1 and a second light guide facility LFE2 of the two light guide facilities may be configured to emit a second light fan LF2. The first light fan LF1 and the second light fan LF2 may be at least rotatable around one rotation axis RA1, RA2 in each instance. The rotation axis RA1 of the first light fan LF1 may intersect a detector surface of the X-ray detector 34 and the rotation axis RA2 of the second light fan LF2 in an operating state of the X-ray device. Advantageously, the X-ray device may be configured to execute a method for controlling an X-ray device.

Advantageously, the X-ray device may also include a control unit CU, which may be configured, in the operating state: to receive REC-PI planning information PI for a planned path P for arranging a medical object MO, which may be imaged for example by the medical X-ray device; to position POS the first defined arrangement in such a way that the rotation axis RA1 of the first light fan LF1 intersects the planned path P; to move MOV the first light fan LF1 and/or the second light fan LF2 in such a way based on the planning information PI and the current positioning MPOS of the first defined arrangement that the first light fan LF1 and the second light fan LF2 illuminate the planned path P.

The control unit CU may also be configured to capture CAP-MPOS the current positioning MPOS of the first defined arrangement in the operating state.

Moving MOV the first light fan LF1 and/or the second light fan LF2 may include at least one rotation of the first light fan LF1 and/or the second light fan LF2 around the respective rotation axis RA1, RA2. The X-ray source 33 may be configured to emit X-rays to illuminate the medical object MO. The control unit CU may be configured to send a signal 24 to the X-ray source 33 for this purpose. The control unit CU may also be configured to send a signal S. LFE1 to the first light guide facility LFE1 and a signal S. LFE2 to the second light guide facility LFE2, wherein the respective signal S. LFE1 and S. LFE2 may actuate the respective light guide facility LFE1 and LFE2 to emit the respective light fan LF1 and LF2. The X-ray detector 34 may be configured to capture the X-rays and to provide a signal 21 to the control unit CU depending on the captured X-rays. The control unit CU may also be configured to provide X-ray image data depending on the signal 21.

An intersection point SP may be defined by the intersection of the rotation axis RA1 of the first light fan LF1 with the planned path P. This intersection point SP may be arranged at a rotation center RZ of the first defined arrangement.

Flexible positioning of the light fans LF1, LF2 may be enabled by this arrangement, in order to guide the placement of a medical object MO along the planned path P. The X-ray device may enable three-dimensional laser guidance for various medical procedures, while simultaneously enabling X-rays to be taken from various angles.

The first and second light fan LF1, LF2 may each be rotatable by a rotatable mounting of the respective light guide facility LFE1, LFE2, and/or an adjustable projection mechanism, and/or an adjustable projection matrix. The rotatable mounting, the adjustable projection mechanism, and/or the adjustable projection matrix may for example be motorized.

The first defined arrangement may be translatably and/or rotatably mounted.

Advantageously, a virtual reference beam RS may be defined as an imaginary line between the X-ray source 33 and the X-ray detector 34. The virtual reference beam RS may thereby run through the rotation center RZ of the first defined arrangement, for example an isocenter of the first defined arrangement. In the form of embodiment shown in FIG. 5, the virtual reference beam RS may be arranged along the rotation axis RA1 of the first light fan LF1. The arrangement of the virtual reference beam RS along the rotation axis RA1 of the first light fan LF1 may thereby include an arrangement deviating slightly from the first rotation axis RA1.

The virtual reference beam RS may be used as a reference line for positioning and orienting the light fans LF1, LF2. For example, the planned path P may intersect the virtual reference beam RS at an intersection point, for example the intersection point SP. This intersection point SP may be used as a reference point for orienting the light guide facilities LFE1, LFE2. Alternatively (not shown here), the intersection point SP may be at any location along the virtual reference beam RS between the X-ray detector 34 and X-ray source 33, depending on the requirements of the specific medical procedure, the potential adjustability of the light guide facilities LFE1, LFE2, and/or mechanical accessibility. This positioning may enable flexible adjustment of the laser needle guidance in various clinical situations.

The control unit CU may be configured to calculate the position of the intersection point SP along the virtual reference beam RS and to use this information to control the light fans LF1, LF2. For example, the control unit CU may orient the rotation axes RA1, RA2 of the light fans LF1, LF2 so that they intersect at the intersection point SP.

The control unit CU may be configured to position the first defined arrangement in such a way that the virtual reference beam RS intersects the planned path P at a fixed intersection point SP relative to the X-ray detector 34. This intersection point SP may be at any location between the X-ray detector 34 and the X-ray source 33, wherein the positioning may be specified by the, for example current, orientation of the rotation axis RA2 of the second light fan LF2. For example, the positioning of the intersection point SP may be preset using the, for example current, orientation of the rotation axis RA2 of the second light fan LF2. Advantageously, the rotation axis RA2 of the second light fan LF2 may be configured as a connecting line between a center of a light-emitting surface of the second light guide facility LFE2 and the intersection point SP at which the planned path P intersects the virtual reference beam RS, for example the rotation axis RA1 of the first light fan. This configuration may enable flexible adjustment of the second light fan LF2 to various path geometries.

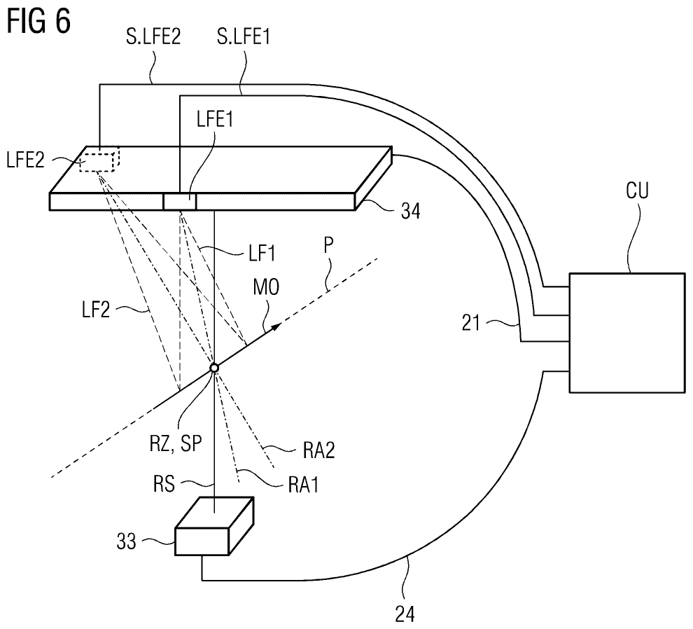

FIG. 6 depicts a schematic representation of an embodiment of a X-ray device. The rotation axis RA1 of the first light fan LF1 may thereby be arranged within the layer that is illuminated by the first light fan LF1. Additionally, the rotation axis RA2 of the second light fan LF2 may be arranged within the layer that is illuminated by the second light fan LF2.

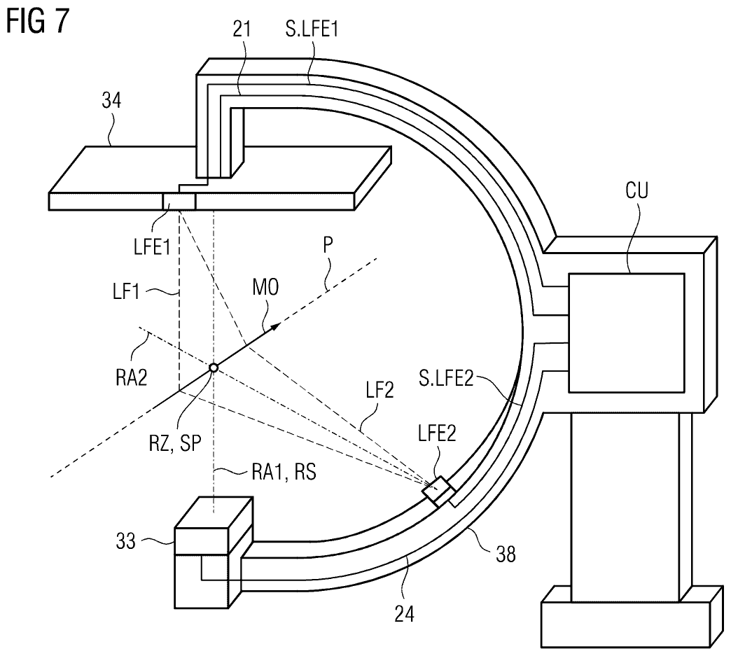

FIG. 7 depicts a schematic representation of another embodiment of a X-ray device. The first light guide facility LFE1 may thereby be arranged in a second defined arrangement with respect to the X-ray detector 34. The second defined arrangement may be rotatably mounted. The rotation axis RA2 of the first light fan LF2 may thereby correspond to a rotation axis of the second defined arrangement.

The X-ray source 33 and the X-ray detector 34 may be arranged in a defined arrangement on a C-arm 38. The C-arm 38 may be movably mounted around one or more axes. For example, the C-arm 38 may be movable in its orientation and/or angulation, and/or translatable in the space, and/or adjustable. Advantageously, the first light guide facility LFE1 may be arranged on the X-ray source 33. The second light guide facility LFE2 may be arranged on a guide unit, for example on the C-arm 38.

The rotation axis RA2 of the second light fan LF2 may, for example, be configured as a connecting line between a center of a light-emitting surface of the second light guide facility LFE2 and an isocenter of the first defined arrangement, for example of a C-arm rotation. The C-arm 38 of the X-ray device may be positioned so that the virtual reference beam RS intersects the planned path at an intersection point SP that is fixed relative to the X-ray detector 34. This may offer a consistent reference for positioning the X-ray device in relation to the planned path P. In the form of embodiment shown in FIG. 7, the virtual reference beam RS may be arranged along the rotation axis RA1 of the first light fan LF1.

After receiving REC-PI the planning information PI, the control unit CU may position the first defined arrangement, for example the C-arm 38, in such a way that a point along the planned path is arranged in the isocenter of the first defined arrangement, for example of the C-arm rotation, and on the virtual reference beam RS. The arrangement in the isocenter or on the virtual reference beam RS may thereby include an arrangement in the proximity of the isocenter or of the virtual reference beam RS, depending on the system inaccuracies and the definition of the virtual reference beam RS. The control unit CU may also be configured to set a desired angulation of the first defined arrangement, for example depending on user requirements. By an appropriate rotation of the X-ray detector 34, the first light fan LF1 may illuminate the planned path P, or the planned path may be arranged in the layer, for example the plane, illuminated by the first light fan. By an appropriate motorized rotation of the second light fan LF2 around its rotation axis RA2, the second light fan LF2 may be oriented in such a way that the planned path P is illuminated by the second light fan LF2 or is arranged in the layer, for example the plane, illuminated by the second light fan. The rotation axis RA2 of the second light fan LF2 may, for example, be configured as a connecting line between a center of a light-emitting surface of the second light guide facility LFE2 to the isocenter of the first defined arrangement, for example of the C-arm rotation. The rotation axis RA2 of the second light fan LF2 may be within the layer of the second light fan LF2 here.

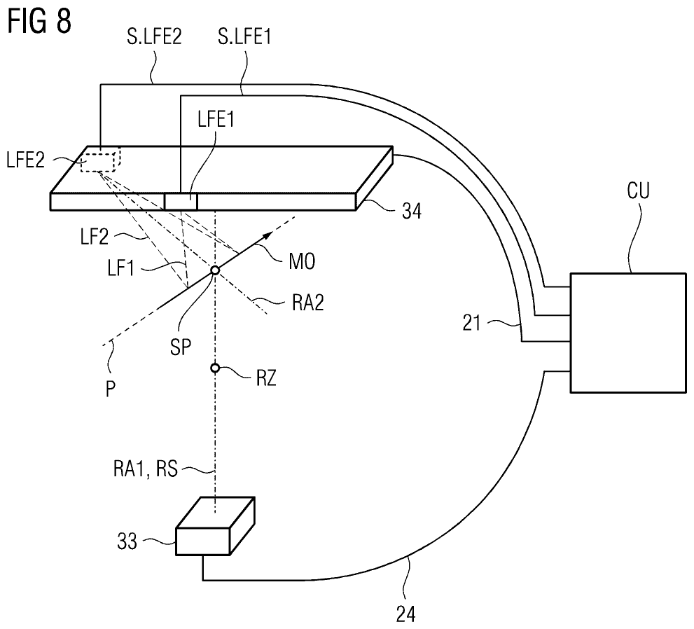

FIG. 8 depicts a schematic representation of another advantageous form of embodiment of a X-ray device. The first and the second light fan LF1, LF2 may thereby also be tiltable by a tiltable mounting of the respective light guide facility LFE1, LFE2, and/or an adjustable projection mechanism, and/or an adjustable projection matrix. The tiltable mounting, the adjustable projection mechanism, and/or the adjustable projection matrix may for example be motorized. The first and/or the second light guide facility LFE1, LFE2 may each be arranged on the X-ray detector 34.

The rotation axis RA1 of the first light fan LFE1 may intersect the rotation axis of the second light fan at the intersection point SP and the detector surface. By tilting both light fans, the intersection point SP may be moved along the rotation axis of the first light fan. This may enable precise positioning of the intersection point SP until the planned path P is illuminated.

In the embodiment shown in FIG. 8, the virtual reference beam RS may be arranged along the rotation axis RA1 of the first light fan LF1. The control unit CU may be configured to position POS the first defined arrangement in such a way that the planned path P intersects the intersection point SP on the rotation axis RA1 of the first light fan LF1, for example on the virtual reference beam RS. By the, for example motorized, automatic, and/or adjustable, tilting of the first and/or second light fan LF1, LF2, the intersection point SP may be chosen, for example may be positioned, freely in an area along the virtual reference beam RS. The, for example motorized, tilting of the second light fan LF2 may be effected in such a way that the, for example motorized, rotation axis RA2 of the second light fan LF2 intersects the desired intersection point SP along the planned path P, for example of a needle axis. Choosing any intersection point SP, for example its positioning, in an area along the virtual reference beam RS therefore only requires a fixed tilting axis of the second light fan LF2 relative to the X-ray detector. If the tilting is set so that the rotation axis RA2 of the second light fan LF2 intersects the intersection point SP, the second light fan LF2 may then be rotated again on the planned path P. The first defined arrangement may be positioned freely as a result of this, because the planned path P does not have to intersect a fixed point relative to the X-ray detector 34, but rather merely a line.

The option of moving the intersection point SP by tilting the light fans LF1 and LF2 along the rotation axis RA1 of the first light fan LF1, may enable a dynamic adjustment of the light guidance. As shown schematically in FIG. 8, the intersection point SP may for example be arranged closer with respect to the X-ray detector 34, than with an arrangement in the rotation center RZ of the first defined arrangement. The advantageous feature of this form of embodiment is that, in many clinical applications, the intersection point SP of the planned path with the virtual reference beam RS is closer to the X-ray detector 34 than the rotation center RZ, for example the isocenter, also due to the space restrictions when positioning the C-arm around a table, an examination object, and/or the medical object MO. It is also advantageous in this case that an angle between the two light fans LF1 and LF2 becomes less acute, for example more obtuse and/or larger, which may enable improved 3D needle guidance by the crossed light fans LF1 and LF2.

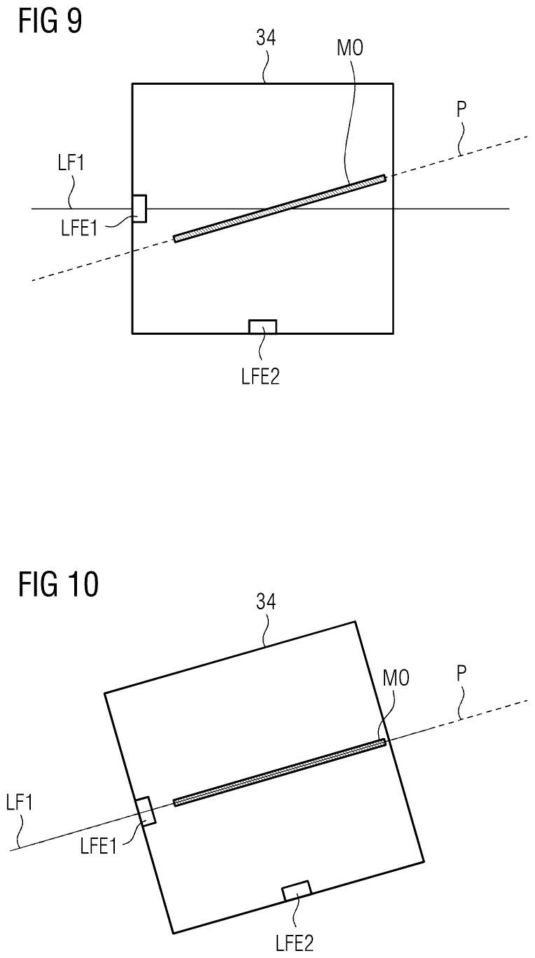

FIGS. 9 and 10 show schematic representations of various positionings of the second defined arrangement. In FIG. 9, the second defined arrangement is shown schematically in a first operating state in an initial positioning. In the first operating state, the first defined arrangement may, for example, be positioned POS so that the rotation axis RA1 of the first light fan LF1 intersects the planned path P. The second defined arrangement may thereby have an initial relative positioning with respect to the first defined arrangement. The first light fan LF1 may thereby be initially positioned in such a way that the first light fan LF1 does not illuminate the planned path P in the first operating state.

In FIG. 10, the second defined arrangement is shown schematically in a second operating state in another positioning. The second defined arrangement may thereby have been moved, for example rotated, in such a way based on the planning information PI and the current positioning POS of the first defined arrangement that the first light fan LF1 illuminates the planned path P. Advantageously, the second light fan LF2 (not shown here) may have been moved in such a way at least in the second operating state that the second light fan illuminates the planned path P.

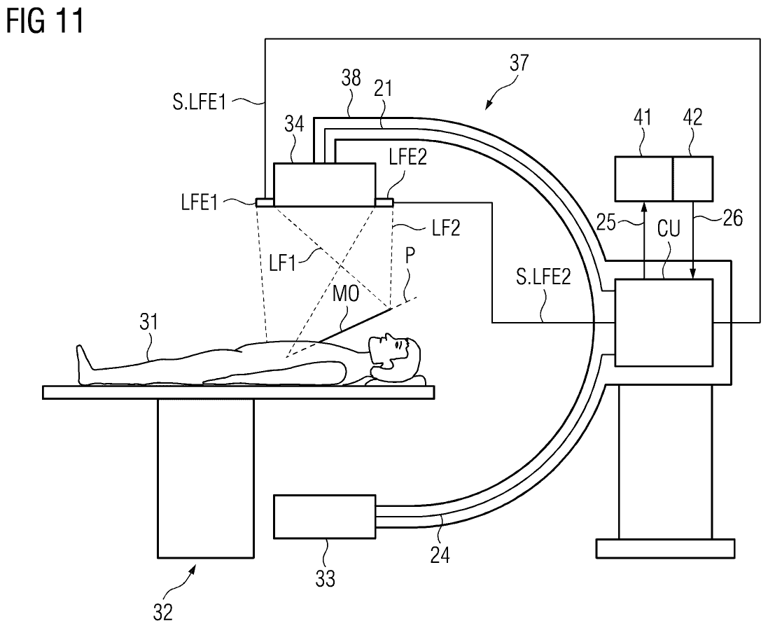

FIG. 11 depicts a schematic representation of another embodiment of a X-ray device. The control unit CU may send the signal 24 to the X-ray source 33. Depending on the signal 24, the X-ray source 33 may then emit X-rays to illuminate, for example to X-ray, the medical object MO and an examination object 31 positioned on a patient positioning apparatus 32. When an X-ray makes contact with an X-ray-sensitive layer of the X-ray detector 34, after interacting with the medical object MO and the examination object 31, the detector 34 may send a signal 21 to the control unit CU. The control unit CU may be configured to capture the X-ray image data using the signal 21.

The X-ray device may also include an input unit 42, for example, a keyboard and/or a joystick, and a display unit 41, for example, a monitor, and/or a display, and/or a projector. The input unit 42 may preferably be integrated into the display unit 41, for example, with a capacitive and/or resistive input display. The display unit 41 may be configured to display a graphic representation of the X-ray image data BD. The control unit CU may send a signal 25 to the display unit 41 for this purpose. The input unit 42 may also be configured to capture a user input. The input unit 42 may also be configured to provide a signal 26 to the control unit CU depending on the captured user input. The control unit CU may be configured to control the X-ray device, for example the positioning of the first defined arrangement and/or the movement of the first and/or second light fan LF1 and LF2, depending on the user input, for example of the signal 26.

For example, repositioning the first defined arrangement into another positioning may be restricted to a rotation around an intersection point SP of the rotation axis RA1 of the first light fan LF1 with the planned path P, a combination including a rotation around the intersection point SP and a translation parallel to the planned path P, or a combination including a rotation around the intersection point SP and a translation parallel to the rotation axis RA1 of the first light fan LF1. This may be effected by adjusting the degrees of control freedom of the input unit 42.

The schematic representations in the described figures are not to scale and do not show proportions accurately.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for controlling an X-ray device, wherein the X-ray device comprises an X-ray source and an X-ray detector opposite each other and two light guide facilities arranged spaced apart from each other, that are arranged in a first defined arrangement;
wherein the first defined arrangement is movably mounted, wherein a first light guide facility of the two light guide facilities is configured to emit a first light fan and a second light guide facility of the two light guide facilities is configured to emit a second light fan, wherein the first light fan and the second light fan are at least rotatable around one rotation axis in each instance, wherein the rotation axis of the first light fan intersects the rotation axis of the second light fan and a detector surface of the X-ray detector, the method comprising:
receiving planning information for a planned path for arranging a medical object;
emitting the first light fan by the first light guide facility;
emitting the second light fan by the second light guide facility;
positioning the first defined arrangement in such a way that the rotation axis of the first light fan intersects the planned path; and
moving the first light fan and/or the second light fan in such a way, based on the planning information and a current positioning of the first defined arrangement, that the first light fan and the second light fan illuminate the planned path;
wherein moving the first light fan and/or the second light fan comprises at least one rotation of the first light fan and/or the second light fan around the respective rotation axis.

2. The method of claim 1, wherein the first light fan and/or the second light fan are also configured as tiltable; wherein moving the first light fan and/or the second light fan further comprises tilting the first light fan and/or the second light fan based on the planning information and the current positioning of the first defined arrangement.

3. The method of claim 1, wherein a focusing of the first light fan and/or the second light fan is adjusted in such a way, based on the planning information and the current positioning of the first defined arrangement, that a respective light fan has a predefined geometry in an area of the planned path.

4. The method of claim 1, wherein the rotation axis of the first light fan is arranged within a layer that is illuminated by the first light fan, and/or wherein the rotation axis of the second light fan is arranged within the layer that is illuminated by the second light fan.

5. The method of claim 1, wherein, repositioning the first defined arrangement into another positioning is restricted to a rotation around an intersection point of the rotation axis of the first light fan with the planned path, a combination comprising a rotation around the intersection point and a translation parallel to the planned path, or a combination comprising a rotation around the intersection point and a translation parallel to the rotation axis of the first light fan;
wherein the other positioning is specified as the current positioning of the first defined arrangement;
wherein the first light fan and/or the second light fan are moved in such a way, based on the planning information and the current positioning of the first defined arrangement, that the first light fan and the second light fan illuminate the planned path;
wherein moving the first light fan and the second light fan comprises at least one rotation of the first light fan and/or the second light fan around the respective rotation axis.

6. The method of claim 1, wherein the rotation axis of the first light fan runs through a rotation center of the first defined arrangement, a geometric center of the X-ray detector, the X-ray source, and/or a geometric center of a radiation exit window of the X-ray source.

7. The method of claim 1, wherein the medical object is arranged along the planned path;
wherein X-rays are emitted by the X-ray source to illuminate the medical object;
wherein the X-rays are captured by the X-ray detector and a signal is provided depending on the captured X-rays;
wherein X-ray image data is provided depending on the signal.

8. The method of claim 1, wherein the planning information is registered with a coordinate system of the X-ray device.

9. An X-ray device comprising:
an X-ray source;
an X-ray detector opposite the X-ray source; and
a first light guide facility and a second light guide facility arranged spaced apart from each other, wherein the first light guide facility is configured to emit a first light fan and the second light guide facility is configured to emit a second light fan, wherein the first light fan and the second light fan are at least rotatable around one rotation axis in each instance, wherein the rotation axis of the first light fan intersects a detector surface of the X-ray detector and the rotation axis of the second light fan in an operating state of the X-ray device;
wherein the X-ray source, the X-ray detector, and the first light guide facility and second light guide facility are arranged in a first defined arrangement that is movably mounted;
wherein the X-ray device is configured to:
receive planning information for a planned path for arranging a medical object;
emit the first light fan by the first light guide facility;
emit the second light fan by the second light guide facility;
position the first defined arrangement in such a way that the rotation axis of the first light fan intersects the planned path; and
move the first light fan and/or the second light fan in such a way, based on the planning information and a current positioning of the first defined arrangement, that the first light fan and the second light fan illuminate the planned path, wherein moving the first light fan and/or the second light fan comprises at least one rotation of the first light fan and/or the second light fan around the respective rotation axis.

10. The X-ray device of claim 9, wherein the first light fan and the second light fan are each rotatable by at least one of a mounting of the respective light guide facility, a projection mechanism, or an adjustable projection matrix.

11. The X-ray device of claim 10, wherein at least one of the mounting or the projection mechanism is rotatable in a motorized manner.

12. The X-ray device of claim 9, wherein the first light fan and the second light fan are each also tiltable by at least one of a mounting, a projection mechanism, or an adjustable projection matrix.

13. The X-ray device of claim 12, wherein at least one of the mounting or the projection mechanism is configured to be tilted using in a motorized manner.

14. The X-ray device of claim 9, wherein the first light guide facility is arranged in a second defined arrangement with respect to the X-ray detector; wherein the second defined arrangement is rotatably mounted; wherein the rotation axis of the first light fan corresponds to a rotation axis of the second defined arrangement.

15. The X-ray device of claim 9 wherein the first light guide facility and/or the second light guide facility are each arranged on the X-ray detector, the X-ray source, or a guide unit.

16. The X-ray device of claim 9 wherein the first defined arrangement is translatably and/or rotatably mounted.

17. A non-transitory computer implemented storage medium, including machine-readable instructions stored therein, the machine-readable instructions when executed by at least one processor, cause the processor to:
receive planning information for a planned path for arranging a medical object;
emit a first light fan by a first light guide facility;
emit a second light fan by a second light guide facility;
position a first defined arrangement of the first light guide facility and the second light guide facility in such a way that a rotation axis of the first light fan intersects the planned path; and
move the first light fan and/or the second light fan in such a way, based on the planning information and a current positioning of the first defined arrangement, that the first light fan and the second light fan illuminate the planned path, wherein moving the first light fan and/or the second light fan comprises at least one rotation of the first light fan and/or the second light fan around a respective rotation axis.
